(19) United States
(12) Patent Application Publication
(10) Pub. No.: US 2003/0216638 A1
(43) Pub. Date: Nov. 20, 2003

(54) Microbubble construct for sensitivity enhanced MR manometry

(76) Inventors: Rohan Dharmakumar, Bolton (CA); Donald Bruce Plewes, Toronto (CA); Graham Arnold Wright, Toronto (CA)

Correspondence Address: DOWELL & DOWELL PC, SUITE 309, 1215 JEFFERSON DAVIS HIGHWAY, ARLINGTON, VA, 22202

(21) Appl. No.: 10/439,325

(22) Filed: May 16, 2003

Related U.S. Application Data
(60) Provisional application No. 60/378,048, filed on May 16, 2002.

(30) Foreign Application Priority Data
Jan. 31, 2003 (CA) 2,418,229

(51) Int. Cl. A61B 5/055

(52) U.S. Cl. 600/420; 424/9.3

(57) ABSTRACT

The present invention provides microbubbles for sensitivity enhanced manometry, and more particularly the present invention relates to a magnetic resonance manometry method for measuring intravascular or intracardiac pressure using microbubbles of high magnetic susceptibility. The invention provides a microbubble for sensitivity enhanced magnetic resonance manometry, comprising a lipid shell having a high magnetic susceptibility. In one aspect the microbubble for sensitivity enhanced magnetic resonance manometry, comprising a lipid shell including magnetic nanoparticles having high dipole moments embedded therein.

### CROSS REFERENCE TO RELATED UNITED STATES PATENT APPLICATIONS

This patent application relates to U.S. Provisional patent application Serial No. 60/378,048 filed on May 16, 2002, entitled MICROBUBBLE CONSTRUCT FOR SENSITIVITY ENHANCED MR MANOMETRY, and Canadian patent application Serial No. 2,418,229 filed on Jan. 31, 2003, entitled MICROBUBBLE CONSTRUCT FOR SENSITIVITY ENHANCED MR MANOMETRY, both published in English, and both patent applications being incorporated herein by reference in their entirety.

### FIELD OF THE INVENTION

The present invention relates to microbubbles for sensitivity enhanced manometry, and more particularly the present invention relates to a magnetic resonance manometry method for measuring intravascular or intracardiac pressure using microbubbles of high magnetic susceptibility.

### BACKGROUND OF THE INVENTION

A quantitative intracardiac pressure measurement can provide clinicians with a strong measure of the functional integrity of the cardiovascular system as noted in E. Braunwald et al. in Heart Disease, p 780-806. It is possible to infer the pressure in the left ventricle with a sphygmomanometer and to date, there have been numerous efforts made to develop a similar non-invasive means of measuring pressure in the right ventricle RV) as noted in M Berger et al. in “Quantitative assessment of pulmonary hypertension in patients with tricuspid regurgitation using continuous wave Doppler ultrasound,” Am J Cardiol 1985; 6:359-365. Such efforts have been made with the intent of replacing widely used catheterization procedures and the associated physical discomfort and risk of infection in patients as noted in D Raeside et al. in “Making measurements in the pulmonary circulation when and how?”, Thorax 1997; 52:9-11. RV pressure measurement using continuous wave Doppler echocardiography based on the peak velocity of the tricuspid jet with the modified Bemoulli's equation is possible only when tricuspid insufficiency exists. However, this usually does not set in until peak RV pressure is greater than 75 mmHg as noted in B J Kircher et al. in “Noninvasive estimation of right atrial pressure from the inspiratory collapse of the lnferior vena cava”, Am J Cardiol 1990;66:493-496. However, the progression of many congenital heart diseases involve small continuous changes in RV pressure. Pulmonary hypertension is defined as an increase in RV systolic pressure above 30 mmHg (or 5 mmHg above the normal systolic pressure of the RV) as defined in Braunwald E et al. Hence, one of the essential requirements of a non-invasive pressure measurement technique is exquisite sensitivity to detect small pressure changes associated with pulmonary hypertension. It has long been realized that distensible micro-bubbles can serve as pressure sensors for non-invasive manometry. Since the 1970's many ultrasound techniques have tried to take advantage of this idea. However, various technical difficulties have prevented their advancement in vivo. A magnetic resonance (MR) based technique that has the potential for detecting intravascular pressure with the aid of a microbubble contrast agent was recently proposed by A L Alexander et al. in “Microbubbles as novel pressure-sensitive MR contrast agents”, Magn Reson Med 1996. 35:801-806. Their hypothesis was based on the observation of earlier reports of other works which showed that the rate of relaxation of the MR signal (R2) from a solution containing spheres (with a susceptibility mismatch relative to the solution) is related to the size of sphere. Since microbubbles respond to pressure changes via volume changes, they correctly predicted that R2 can be used to calibrate and serve us a pressure marker in vivo. While the early experimental results in vitro have shown this successfully, an in vivo use of this technique for early detection of pulmonary hypertension (25 mmHg above right ventricular systolic pressure or 50 mmHg above atmospheric pressure) is currently limited by inadequate sensitivity.

The current limitations of microbubble based MR manometry are as follows,

(1) Inadequate R2 measurement accuracy associated with detecting small changes in pressure. The measurement errors in R2 originate from cardiac motion, breathing, flow depbasing, and partial volume effects.

2) Suboptimal changes in R2 for a given pressure change in the presence of microbubbles in the blood stream for a given microbubble dose. In the presence of microbubbles in vivo,

R2^{Blood}≈R2^{Diss}+R2^{RBC}+R2^{Bubb}, (1)

where R2^{Diss} is the rate constant associated with the decay of the MR signal due to dissipative mechanisms such as dipole-dipole coupling and is ˜4 s⁻¹. R2^{RBC} and R^{Bubb} are the rate constants connected with decay of the MR signal due diffusion of spins in a field gradient set up by the red blood cells and the bubbles respectively. At 1.5T, with a refocusing interval (τ₁₈₀) of 6 ms at oxygenation saturation of 70% O₂ in the pulmonary trunk is R2^{RBC}˜1.2 s⁻¹. Hence, under similar conditions, we anticipate that for the bubbles to dominate the relaxation process, Cobb should be at least 5 s⁻¹.

3) Even large pressure changes (100's mmHg) cannot be detected without physiologically toxic microbubble dose. Toxicity testing of microbubble formulations to date has shown that, when the dose of bubble formulations exceed 1 cc/kg, clinical complications emerge. The volume fraction of gas used by Alexander et al. corresponds to a dose of approximately 3 cc/kg (assuming 4 L of blood in a 70 kg body) that would be toxic in vivo. This means that a large enough R2^{Bubb} needs to be established in vivo with the smallest possible microbubble dose. Preliminary experiments at 4.7 T by others have revealed that, when microbubbles containing air (mean radius of 3.03±0.53 m) are used 3 cc/kg, the spin echo R2^{Bubb} is 18 s⁻¹, which is large enough to produce the adequate sensitivity. However, considering the target clinical utility, the low pressure sensitivity of R2, toxic microbubble doses are required. In addition, at large static field strengths (such as 4.7T) the contribution of R2^{RBC} to R2^{Blood} will also be higher. In addition, at high fields it is anticipated that the motion and flow artifacts will further degrade the accuracy of the measurement technique.

As shown by R Dharmakumar et al., in “On the parameters affecting the sensitivity of MR measures of pressure with microbubbles”, MRM 2002, 47: 264 273 previously there are a number of parameters that affect the sensitivity of microbubble based MR manometry. Results show that the MR sensitivity to pressure changes is strongly dependent on the bubble size at atmospheric pressure (R₀), static magnetic field strength, magnitude of the susceptibility difference between the encapsulated gas and plasma (Δ₁₀₂), and bubble volume fraction. It was also found that the optimum bubble size is strongly dependent on the type of nuclear magnetic resonance (NMR) measurement method and improves with increase in magnetic field strength, susceptibility difference, and volume fraction. To reduce measurement errors associated with detecting MR signal for cardiovascular applications it has been suggested that Carr-Purcell-Meiboom-Gill based pulse sequence is used by G A Wright et al., in “Estimating oxygen saturation of blood in vivo with MR imaging at 1.5T”. JMRI 1991;1:275-283. In addition, given that most common commercial MR scanners operate at 1.5 T and physiological complications based on microbubble toxicity arise when the dose exceeds 1 cc/kg of body weight it was concluded that for R2^{Bubb} to be larger than 5 s⁻¹, R₀ should be 2-3 μm and Δ₁₀₂≧34 PPM (SI units). Although optimum R₀ is feasible, the microbubble contrast agent with the largest realizable Δ_{χ} that is limited by the inherent low density of gases at 11 ppm (SI units). Hence the successful clinical implementation of MR manometry relies on improved susceptibility difference in excess of 34 ppm, physiologically tolerable microbubble doses, a calibration scheme to relate pressure changes to R2, and an MR protocol to make the requisite measurement.

### SUMMARY OF THE INVENTION

The inventors show that a specialized microbubble design can effectively increase the Δ_{χ} to desired levels through enhacing the magnetic susceptibility of microbubble shell. In particular they show that embedding magnetic nanoparticles of high dipole moment on the lipid shell of the typical microbubbles can increase the Δ_{χ} to desired levels while preserving the pressure sensitivity of microbubbles in the MR field. This is shown by first re-deriving the governing equation of field perturbation around a gas containing bubble coated with a highly susceptible continuous shell. From there it is shown that the continuous shell case is equivalent to uniformly coating the lipid shell with particles of high dipole moment. It is disclosed herein that the resulting Δ_{χ} is a function of particle dipole moment, size and density on the shell. In addition, with the aid of Monte Carlo simulations they show that when particles of high enough dipole moment are coated at low volume fraction, it is feasible to elevate R2^{Bubb} well beyond 5 s⁻¹. It is disclosed that microbubble dose is proportional to R2^{Bubb} and the present invention establishs that by controlling the volume fraction of the particles on the microbubble shell it is also possible to reduce the microbubble dose within a physiologically acceptable range. Through the theoretical work underpinning the present invention it is demonstrated that these specialized microbubbles are capable of acting as highly sensitive non-invasive pressure probes that will be instrumental in the sensitive detection of moderate pulmonary hypertension with magnetic resonance imaging. The present invention also shows how this technique may be implemented from the fabrication of the necessary microbubbles to the MR protocol to measure pressure.

In addition to detecting pulmonary hypertension in vivo, this technique may also be broadened to measure intracardiac and intravascular pressure anywhere else in the circulation. For instance, using this technique one should be able to measure aortic pressures that are not visible to sphygmomanometer, pressure changes in atheroscelortic regions of the vasculature, intracranial pressure, and pressure in the ocular cavity to name a few.

The present invention provides a microbubble for sensitivity enhanced magnetic resonance manometry, comprising a lipid shell having a high magnetic susceptibility.

The present invention provides a microbubble for sensitivity enhanced magnetic resonance manometry, comprising a lipid shell including magnetic nanoparticles having high dipole moments embedded therein.

The present invention provides a microbubble for sensitivity enhanced magnetic resonance manometry, comprising a lipid shell including a magnetically active agent attached to, or incorporated into, the surface of the bubble to give said microbubble a pre selected magnetic susceptibility.

In another aspect of the present invention there is provided a magnetic resonance imaging method for measuring intravascular or intracardiac pressure in a patient, the method comprising the steps of;

a). intravenously administering microbubbles to a patient, said microbubbles comprising a lipid shell having a high magnetic susceptibility;

b). performing cardiac-gated, flow and/or motion compensated magnetic resonance imaging to establish microbubble concentration dependent and pressure independent magnetic resonance (MR) signal decay in a major blood vessel or in a sample of blood drawn from said patient; and

c). measuring decay of the magnetic resonance signal in a region of interest in the patient's body, comparing a difference between pressure independent magnetic resonance signal decay and pressure dependent magnetic resonance signal decay to a calibration curve between magnetic resonance signal decay and pressure to determine the pressure in the region of interest.

### BRIEF DESCRIPTION OF THE DRAWINGS

The method of the present invention will now be described, reference being had to the accompanying drawings, in which:

FIG. 1 is a schematic representation of a sphere with inner radius of Rᵢₙ and outer radius Rₒᵤₜ and a uniform external magnetic field of H₀ directed along the z-axis. The magnetic permeability of the region outside the sphere is μ₀, in the shell is μ₁, and of the gas inside the sphere is μ₂;

FIG. 2 is a plot showing the relationship between the shell thickness and effective magnetic susceptibility difference between the fluid and the gas containing microbubble with shells of non-negligible magnetic permeability;

FIG. 3 is a plot showing the relationship between the susceptibility of particles of different sizes embedded on microbubble shell and the effective magnetic susceptibility difference between blood plasma and air containing microbubble. In all cases microbubble radius was fixed at 2 μm;

FIG. 4 is a plot showing the relationship between the two ways of increasing the particle volume shell fraction and the effective magnetic susceptibility difference between blood plasma and air containing microbubble. In both cases particle susceptibility was fixed at 10000 πppm; and microbubble radius was fixed at 2 μM;

FIG. 5 is a plot comparing the effect of pressure changes on R₂ of blood containing free air bubble and magnetite coated microbubble. Both. microbubbles are 2 μm in radius and contain air in their lumen. The air bubble volume fraction was 0.1527% (or dose of 0.87 cc/kg) but the magnetite coated microbubble volume fraction was 0.0344% (or a dose of 0.2 cc/kg). Magnetite particles were 15 nm in radius and had a total magnetic susceptibility of 128000 πppm. R₂ was obtained via Monte Carlo simulations with a refocusing interval=6 ms; diffusion coefficient of water 2.75×10⁻⁹ m²·s⁻¹; B₀=1.5T; and

FIG. 6(A-C) show the different microbubble constructs with magnetically active agents that can be prepared: agents incorporated onto the surface (A), in between the bilayers (B), or within an oil layer of a multilamellar structured microbubble (C).

### DETAILED DESCRIPTION OF THE INVENTION

I. A Continuum Model

When a spherical microbubble is placed in a fluid with a magnetic permeability of μ₀ in which an external uniform magnetic field H₀ is present, the field around the microbubble is disturbed. The equation which represents this field is the solution to the associated 3D Laplace equation of the magnetic scalar potential, A. If we let μ₂ to represent the magnetic permeability of gas inside the bubble, μ₁ represent the magnetic permeability of the shell of the micro-bubble, and H represent the magnetic field intensity and B represents the magnetic field (where B=μH) then the Maxwell's equations corresponding to this magnetostatic can be combined in a 3D Laplacian for magnetic scalar potential given by

∇²A=0, (2)

which is a well known partial differential equation which has the following solution in spherical coordinates (r,θ,φ):

A₀=(−H₀r+I₀/r²)cos θ,r>R₁

A₁=(−H₁r+I₁/r²)cos θ,R₁>r>R₂

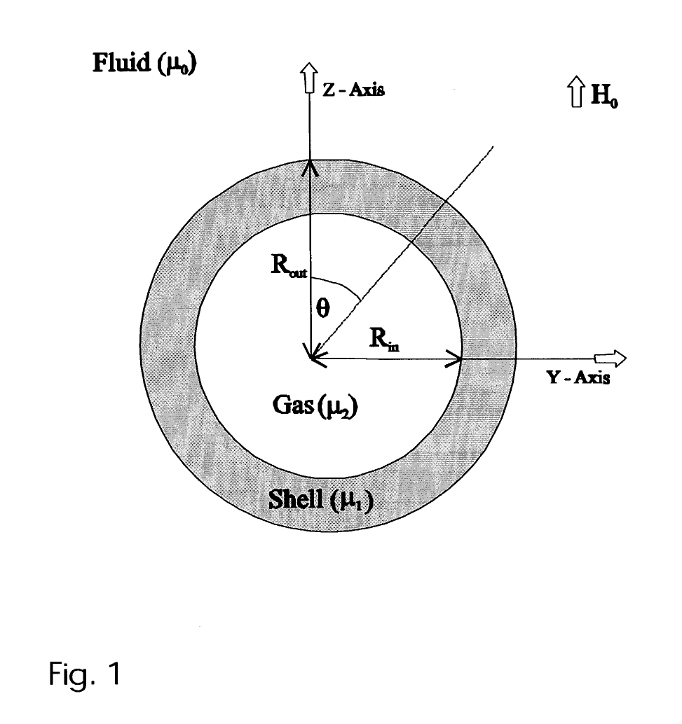

A₂=H₂r cos θ,r<R₂, (3)

where A₀, A₁, A₂ correspond to the magnetic scalar potential outside the bubble, in the shell, and inside the bubble respectively and I₁, H₁, and H₂ are to be solved from the boundary conditions. Rᵢₙ is the radius of the bubble without the shell and Rₒᵤₜ is the radius of the bubble with the shell (refer to FIG. 1).

The boundary conditions for the problem must satisfy the following criteria: the tangential component of H (or H_{θ}) and the radial (or normal) component of B (or B₀) are continuous across the different boundaries [9]. From here, it is possible to show that the z component of the local field perturbation in the vicinity of the bubble in terms of the dipole moment, α, in the spherical coordinate system is given by

$$\begin{matrix}Δ {B}_{x} = \frac{α}{{r}^{3}} \cdot ( 3 {cos}^{2} θ - 1 ) , \\ where & ( 4 ) \\ α = \frac{9 {μ}_{1} {μ}_{0} Δ {χ}_{2} {B}_{0} {R}_{i n}^{3}}{( {μ}_{1} + 2 {μ}_{0} ) [ ( 2 {μ}_{1} + {μ}_{2} ) ( 2 {μ}_{0} + {μ}_{1} ) - 2 Δ {χ}_{2} \cdot Δ {{χ}_{1} ( \frac{{R}_{i n}}{{R}_{out}} )}^{3} ]} + & \\ \frac{Δ {χ}_{1}}{{μ}_{1} + 2 {μ}_{0}} {B}_{0} {R}_{out}^{3} , & \end{matrix}$$

with Δ_{χ1}=μ₁−μ₀; Δ_{χ2}=μ₂−μ₁; B₀=μH₀; μ=1+χ, where X the magnetic susceptibility.

(A). Shell-Free or Lipid-Shelled Gas Bubble in Plasma

When there is no shell present (Rₒᵤₜ=Rᵢₙ) or when the shell is made of lipid bilayer (μ₁˜μ₂), the first term in the above equation vanishes and we get

$$α = \frac{Δ {χ}_{1}}{{μ}_{1} + 2 {μ}_{0}} {B}_{0} {R}_{out}^{3} . \qquad ( 5 )$$

Moreover if the magnetic susceptibility of the fluid and the gas are very small then μ₀˜μ₁≈1 and hence

$$α = \frac{Δ {χ}_{1}}{3} {B}_{0} {R}_{out}^{3} . \qquad ( 6 )$$

Thus from Eq. (4), it follows that the field perturbation along the z-axis is

$$Δ {B}_{z} = \frac{1}{3} \cdot Δ {χ}_{1} {B}_{0} \cdot {( \frac{{R}_{out}}{r} )}^{3} \cdot ( 3 {cos}^{2} θ - 1 ) . \qquad ( 7 )$$

This is the equation originally reported by J A Glasel et al in “On the interpretation of water nuclear magnetic resonance relaxation times in heterogeneous systems”. J Am Chem Soc 96:970 (1974). These were the same equations that were used by R Dharmakumar et al with lipid-shelled microbubbles. Under these conditions the only way to change the magnetic susceptibility of such bubbles in the plasma is to charge the susceptibility of the encapsulated gas.

(B). Gas Bubble with Highly Susceptible Shell in Plasma

Now suppose the sphere has a shell that is highly permeable to the static magnetic field. If the susceptibility of this shell is much larger than the gas inside the bubble or fluid outside the bubble (or χ₂<<χ₁ and χ₀<<χ₁), the magnetic dipole moment can be reduced to

$$\begin{matrix}α = \frac{1}{3} \cdot Δ {χ}_{eff} \cdot {B}_{0} \cdot {R}_{out}^{3} , \\ where & ( 8 ) \\ Δ {χ}_{eff} = [ \frac{3}{3 + {χ}_{1}} ] \cdot [ Δ {χ}_{1} + \frac{9 \cdot ( 1 + {χ}_{1} ) \cdot Δ {χ}_{2} \cdot {β}^{3}}{( 2 Δ {χ}_{1} + 3 ) ( {χ}_{3} + 3 ) + 2 {χ}_{1}^{2} \cdot {β}^{3}} ] & ( 9 )\end{matrix}$$

and denote Rᵢₙ=βRₒᵤₜ where βε(0,1), and μ₁=1+χ₁. From Eq. (4), it follows that the field perturbation along the z-axis is

$$Δ {B}_{z} = \frac{1}{3} \cdot Δ {χ}_{eff} \cdot {B}_{0} \cdot {( \frac{{R}_{out}}{r} )}^{3} \cdot ( 3 {cos}^{2} θ - 1 ) \qquad ( 10 )$$

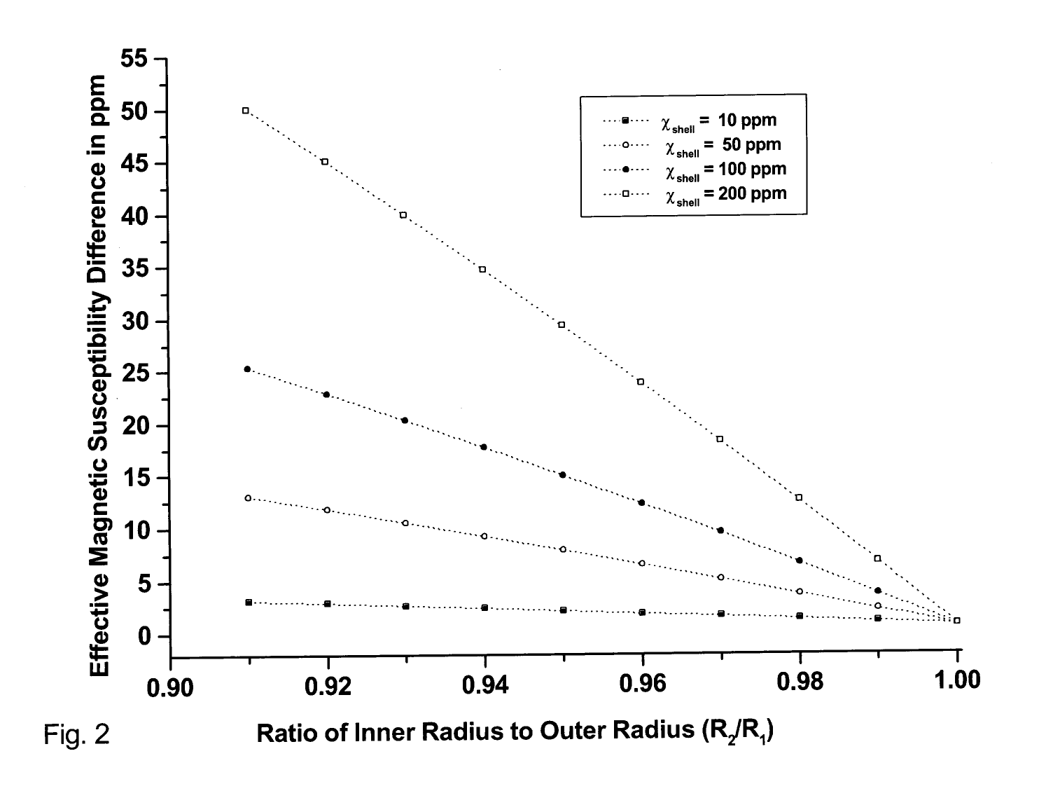

The above equation is quite appealing since it is identical in form to the shell-free equation we used in our earlier work but now with Δ_{χ}=Δχ_{eff}(β, χ₀, χ₁, χ₂) The dependence of Δχ_{eff} on shell thickness and shell susceptibility is shown in FIG. 2. From this figure it is clear that increasing the shell thickness or increasing the magnetic susceptibility of the shell is equivalent to improving the susceptibility difference between the bubble and its environment,

A Discrete Model

In practice one means of enhancing the microbubble shell susceptibility is by coating or embedding magnetically active particles of high magnetic dipole moment. Using finite element analysis, we modeled the field perturbations around the bubble as function of particle size (Rp), density (Ω), and total magnetic susceptibility (χ_{Tot}) Finite element analysis was performed with the aid of Maxwell 3D(Ansoft Corp, Pittsburgh, USA). The finite element consisted of mesh volumes of no larger than 0.05 μm³; at this meshing the theoretical prediction of the field surrounding a free bubble agreed to more than 99% with the fields computed with Maxwell 3D. One eighth of a sphere of 2 μm radius was placed at one of the corners of a cube of 15 μm length. On this sphere, spherical particles of radius of known size and χ_{Tot} were uniformly distributed at different densities. Exploiting the symmetry in the model and taking the inside of the bubble to contain air and the outside to be blood plasma, the fields surrounding the bubbles was computed on a PC with AMMD Athlon XP 1600+ processor (Sunnyvale, Calif.). The field patterns surrounding the bubble were then used to fit Eq. [10] and Δχ_{eff} was computed.

(A). Effect of Rp anA χ_{Tot} on ΔX_{eff}

To study the effect of particle size on Δχ_{eff}, particle size was varied from 5 nm to 30 nm in interval of 5 nm while keeping the particle density fixed at a solid angle of approximately 5°. To ensure Maxwell's program correctness criteria, all overlapping particles on the surface of the sphere were eliminated. To study the effect of χ_{Tot} on Δχ_{eff}, while keeping all other parameters the same, χ_{Tot} was changed incrementally by the following sequence 400 π ppm, 2000 πppm, 4000 πppm, 6000 πppm, 8000 πppm, 10000 πppm.

(B). Effect of Ω on Δχ_{eff}

To study the effect of particle density on the effective magnetic susceptibility, Rp and χ_{Tot} were fixed at 15 nm and 10000 πppm respectively and Ω was varied from 3.75° to 15° with

TABLE 1
Magnetic properties of naturally available minerals

| Mineral | R_{SD}(nm) | ${χ}_{Tot}^{a} ( \times {10}^{3} ppm )$ | Density (g/cm³) |
|---|---|---|---|
| Iron (Fe) | 4-13 | 1400 | 7.874 |
| Magnetite (Fe₃O₄) | 12-30 | 400 | 5.191 |
| Maghemite (γ-Fe₃O₄) | 5-30 | 320 | 5.074 |
| Hematite (α-Fe₃O₄) | 13-7500 | 2.1 | 5.271 |

^{a}χ_{Tot} is the total magnetic susceptibility at 1.5 T
Ω ∈ (3.75°, 5°, 7.5°, 9°, 15°).

From the theoretical results so fax inventors foresee that any magnetic particle of any size that can positively enhance the Δχ_{eff} can be attached to the microbubble would enhance the sensitivity of magnetic resonance imaging based manometry. In nature there are many such particles and in Table 1 inventors list a few such particles with their physical and magnetic properties as reported by D J Dunlop et al in Rock magnetism: fundamental and frontiers. Cambridge University Press, 1997. p.51 and 131.

(C). Effect of Changing Pressure on R₂ with Coated Microbubbles

To study the effect of changing pressure with microbubbles coated with magnetically active particles, inventors simulated the experiment with microbubbles (R₀=2 μm) coated with single domain magnetite particles at Ω=5° and χ_{Tot}=0.128 π. Assuming the isothermal pressure volume relationship holds, the bubble size at pressure (P₀+ΔP) was computed according to

$$R = {( \frac{{p}_{0}}{{P}_{0} + Δ P} )}^{1 / 3} \cdot {R}_{0} ,$$

where P₀ is the atmospheric pressure and ΔP is the incremental change in pressure above P₀. By keeping Ω=5° and χ_{Tot}=0.128 π fixed and varying the bubble size by changing ΔP in increments of 50 mmHg from 0 mmHg up to 150 mmHg, Δχ_{eff} was computed via finite element analysis assuming the inside of the bubble is composed of air and outside of the bubble was blood plasma. This data were used in conjunction with a Monte Carlo simulation as performed earlier and the relation between pressure and R₂(CPMG) was computed. The parameters for the Monte Carlo simulations were: (1) Diffusion coefficient of water in blood plasma=2.75×10 ⁻⁹ m²·s⁻¹; (2) bubble volume fraction=0.0344% corresponding to a dose 0.2 cc/kg; (3) τ₁₈₀=6 ms; (4) time step of protons=10 μs; (5) number of protons=10⁴; (6) B₀=1.5T.

(D). Simulation Results

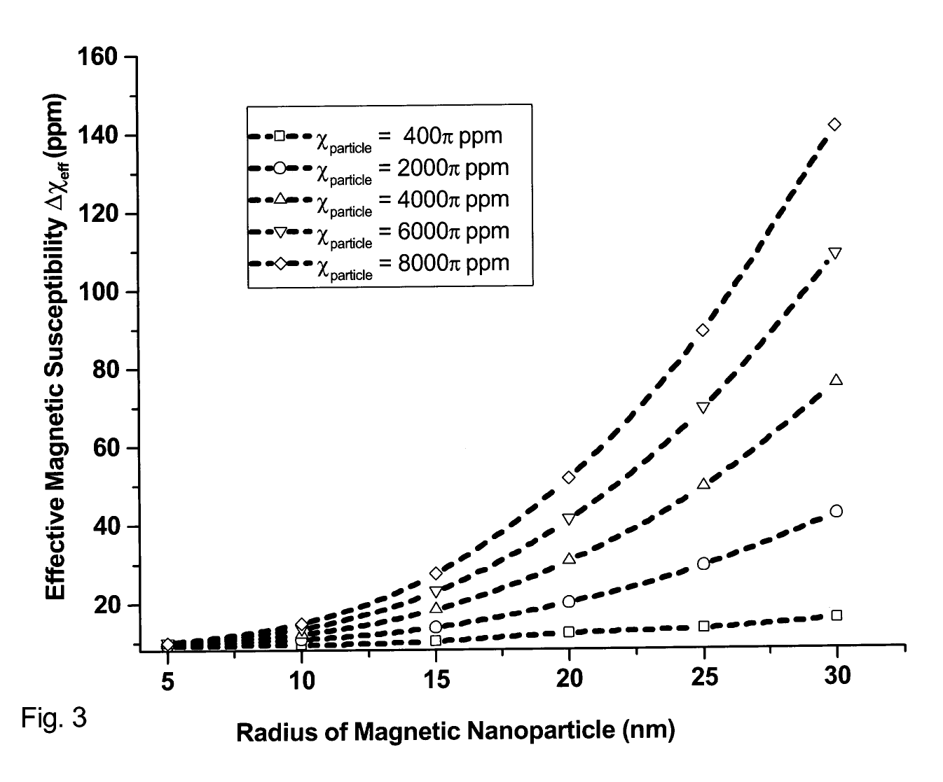

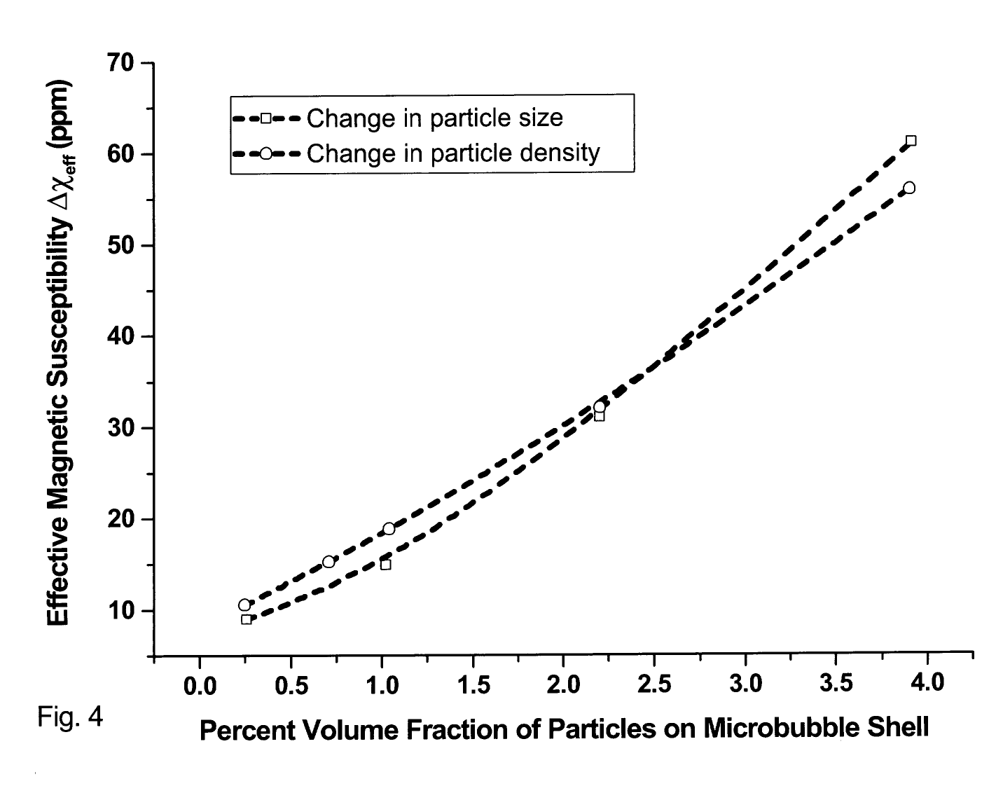

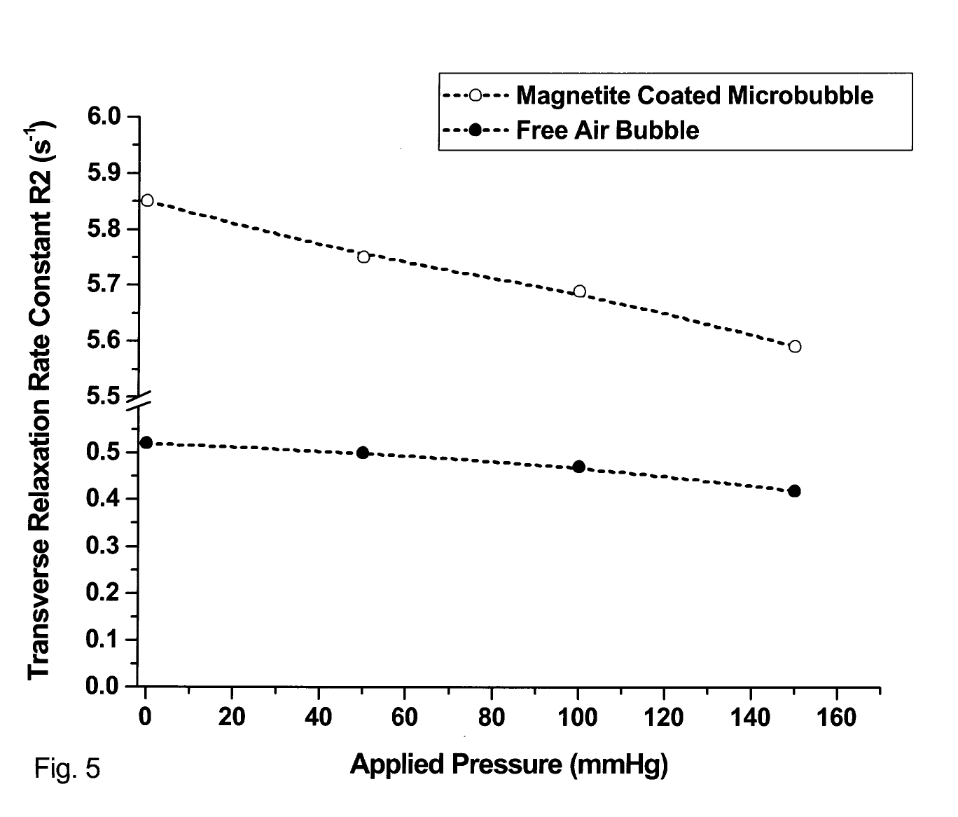

Increasing the magnetic susceptibility of the spherical inclusions on the microbubble shell or their size, monotonically increases the overall effective magnetic susceptibility between the bubble and its environment. This is shown in FIG. 3. It is also evident from this figure that the dependence between particle size and Δχ_{eff} is nonlinear and dΔχ_{eff}/dRp is much smaller at small particle sizes such as 5 nm compared to larger particle sizes such as 20 nm. This implies that for a given number of particles distributed at a known surface density, Δχ_{eff} can be more effectively increased with larger particles than smaller ones. In addition, increasing the particle density also increases the Δχ_{eff}. The effect of increasing particle density is related to increasing particle size as they both contribute to increasing the microbubble shell volume fraction of the particles where shell volume fraction (VFₛₕₑₗₗ) is defined as the composite volume of all the particles on the microbubble shell divided by the volume of the microbubble shell or

$$V {F}_{shell} = \frac{n}{6} \cdot {[ \frac{{R}_{p}}{R} ]}^{2} , \qquad ( 11 )$$

where n is the number of particles on microbubble surface and R is the size of the bubble (refer to Appendix B). This implies that changing the density changes n and changing the particle size changes Rp. Using this common parameter we show the effect of changing volume fraction on Δχ_{eff} by changing particle size and particle density in FIG. 4. From this figure it is evident that, at low VFₛₕₑₗₗ, Δχ_{eff} could be improved by increasing density. However, beyond a critical point, improvement in Δχ_{eff} is best achieved by increasing particle size. Finally, the effect of changing pressure in the presence of coated microbubbles and shell-free bubbles containing air on multi-echo R₂ is shown in FIG. 5.

It is clear that pressure sensitivity is improved with coated microbubbles in comparison with air bubbles. However, unlike susceptibility enhancing contrast agents as suggested ill U.S. Pat. Nos. 5,215,680, 5,088,499, 6,416,740, and the world pat No. WO09851284 these must be fabricated so that their ability to report pressure and hence their compressibility in relation to conventional magnetic agent-free microbubbles is not adversely altered. In the manufacturing process the compressibility of the shells can be studied with the ultrasound scattering properties can be used to study the compressibility changes in the shell due to the loading of magnetically active agents.

Fabrication of Pressure Sensitive and Shell Susceptibility Enhanced Medical-Grade Microbubbles

Magnetically active agents not limited to paramagnetic, superparamagnetic, or ferromagnetic origin or their variations may be incorporated on the surface, as transmebrane structures, or embedded in a compartment of a multilameller lipid-shelled microbubble construct in a number of ways. The methods listed below can serve as a means of preparing the desired construct for sensitivity enhanced MR manometry.

(A). Monolayered Lipid-Shelled Microbubbles Externally Coated with Lanthanide Trivalent Metalic Complexes

As disclosed in U.S. Pat. No. 5,215,680 which is incorporated herein by reference, the prior art fabrication is performed in a two step process where the medical grade lipid-shelled microbubbles are produced and then are subject to subsequent paramagnetic labeling.

(i) Formation of Medical Grade Lipid-Shelled Microbubbles

A surfactant mixture with a preferred composition of Glyerol Monolaurate, Cholesterol Benzoate, Cholesterol, Cholesterol Acetate, and Glycerol Tripalmitate is formed by admixing the agents in a weight ratio of 3:1:1:1:1. A saturated lipid emulsion is obtained when this surfactant mixture is mixed in saline solution (0.02 to 0.4 g of sufactant mixture: 100 cc of saline). The resulting mixture is shaken vigorously for 10 seconds in air or other gaseous material at room temperature. After 5 min, shaking is repeated 2 or 3 more times. Following the shaking, the solution is allowed to stand for 30 ml so that the undissolved material settles out of solution. The resulting solution is filtered through a polysulfone membrane filter [Gelman Sciences, Ann Arbor, Miss.] with average pore diameter of 0.45 μm. Particle sizing can be performed with electroimpedance-sensed volumetric sizing with Coulter Multisizer with Coulter's Accucomp data handling software. The expected characteristics is as follows: maximum bubble diameter of 6 μm (mean diameter of 2 μm, 99% below 4.5 μm in diameter) at a particle density of 540,000±15% per mL.

(ii) Labeling the Monolayered Microbubbles with a Desired Paramagnetic Label

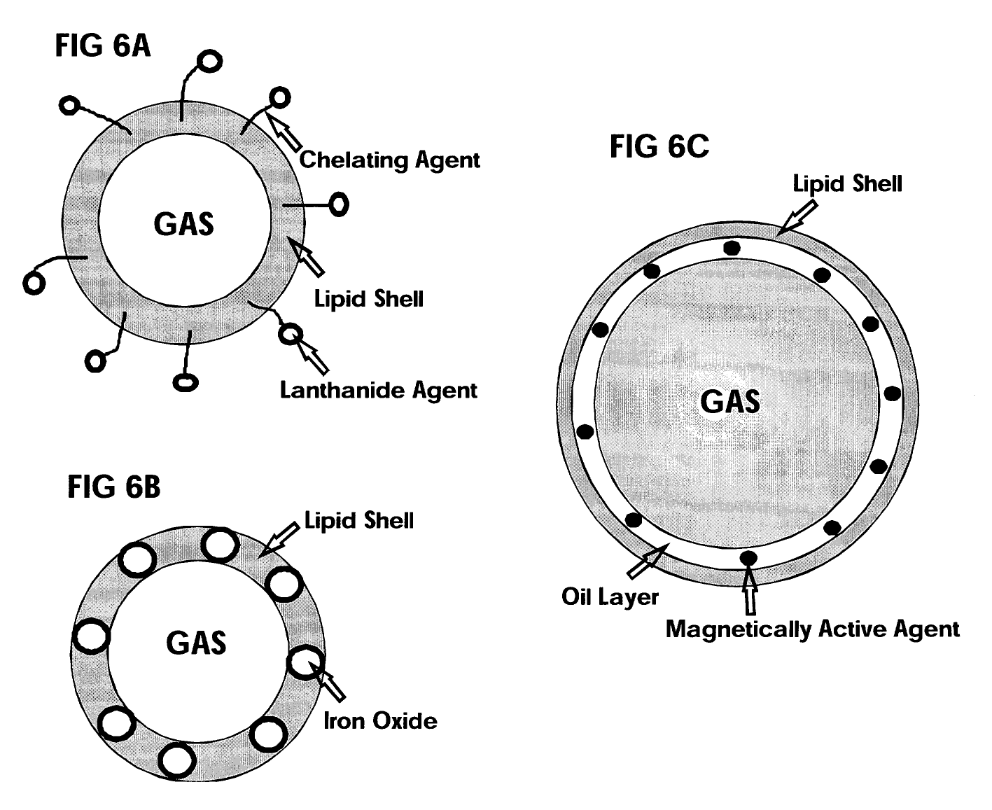

The surface active paramagnetic label is obtained as a lyophilized powder. Dissolve 15 g of polyalanine (a moderately hydrophobic, neutral amino acid copolymer of 1000-5000 daltons in molecular weight) in 2500 mL of 1.0 M phosphate buffer and filter through 0.5 μm pore-diameter filter. Add 20 fold molar excess of solid DTPA [Sigma Chemical, St. Louis, Mo.] to protein solution and adjust pH to 8 by adding sodium phosphate buffer. Stir for 30 min and lower the pH to 5.6 by adding glacial acetic acid (or concentrated HC1). Add 30 fold molar excess of GDCl₃ [Aldrich Chemical Co, Milwaukee, Wis.] to protein. Perform dialysis with the solution against 0.15 M saline at 5·for 96 hours using 1000-Dalton cutoff dialysis tubing. Lyophilize the resulting 1.8-2.0L solution over several days to give white, solid derivative (˜20 g). In order to incorporate Gd-DTPA derivative into lipid-coated microbubbles the powdered derivative needs to be combined with the lipid-sufactant mixture used to form the microbubbles at 5-10% w/w. When this mixture is shaken in isotonic saline, paramagnetic labelled surface active derivative is incorporated into microbubble's surrounding lipid monolayer with Gd-DTPA remaining exposed to aqueous exterior. Refer to FIG. 6A.

(B). Lipid-Shelled Iron Oxide Encapsulated in Oligolamellar Microbubbles

As disclosed in U.S. Pat. No. 5,088,499 which is incorporated herein by reference, the prior art fabrication is performed by incorporating iron oxide particulates internalized by pre-formed liposomes via base catalysis.

(i) A. Liposome Construction

The lipids used may be of either natural or synthetic origin. Such materials include, but are not limited to, lipids such as cholesterol, phosphatidyleholine, phosphatidylethanolarine, phosphatidylserine, phosphatidylglycerol, phosphatidicacid, phosphatidylinositol, lysolipids, fatty acids, sphingomyelin, glycosphingolipids, glucolipids, glycolipids, sulphatides, lipids with ether and ester-linked fatty acids, polymerizable lipids, and combinations thereof The liposomes may be PG, 17 synthesized in the absence or presence of incorporated glycolipid, complex carbohydrate, protein or synthetic polymer, using conventional pro cedures. The surface of a liposome may also be modified with a polymer, such as, for example, with polyethylene glycol (PEG), using procedures readily apparent to those skilled in the art. Any species of lipid may be used, with the sole proviso that the lipid or combination of lipids and associated materials incorporated within the lipid matrix should form a bilayer phase under physiologically relevant conditions. The composition of the liposomes may be altered to modulate the biodistribution and clearance proper-ties of the resulting liposomes. To incorporate ionophores into the liposome membrane, the ionophores, which are lipophilic, are simply added to the lipid mixture, and the liposomes are prepared in the usual fashion. In addition, the size of the vesicles can be adjusted by a variety of procedures including filtration, sonication, homogenization and similar methods to modulate liposomal biodistribution and clearance. To increase internal aqueous trap volume, the vesicles can be subjected to repeated cycles of freezing and thawing. The liposomes of the invention may be of varying sizes, but preferably have a mean outer diameter between about 30 nm and about 10 μm.

(ii) Internalizing Iron Oxide Magnetite

One method of entrapping a particulate solid contrast enhancing agent such as magnetite, within an existing liposome is via base catalysis. Ii this method, a mixture of ferrous and ferric salts is entrapped within the aqueous core of the liposome containing a gaseous precursor. An ionophore such as valinomycin is incorporated within the matrix of the liposome in order to increase the rate of proton flux across the membrane. Prior to or during use, the pH on the exterior of the vesicle is then increased by the addition of the appropriate alkali resulting in an increase in the pH in the interior of the liposome. The increase in pH in turn promotes base catalysis which results in the in situ formation of highly susceptible magnetite within the liposome. It is equally possible to entrap preformed solid contrast enhancing agents such as preformed magnetite in the liposomes. The magnetite containing microbubbles with gaseous precursor can then be converted to gas containing microbubbles via change in pH in the internal environment, exposure to UV light, or increased temperature. In this formulation, the iron oxide magnetite is incorporated into the walls of the lipid-based microbubbles. Refer to FIG. 6B

(C). Lipid-Shelled Magnetically Active Agents Internalized in the Oil Layer of a Multilammelar Microbubble

As disclosed in World Pat. No. WO09851284 and U.S. Pat. No. 6,416,740 which are incorporated herein by reference, the prior art fabrication is performed by incorporating any magnetically active agent in the oil layer of the multilamellar microbubble construct. Such a microbubble contrast system will comprise of an oil, surfactant, a magnetically active agent complexed with a lipophillic agent, and a gas. Magnetically active elements such as Gd(III), Mn(II), Cu(II), Cr(II), Fe(II), Fe(III), Co(II), Er(II), Ni(II), Eu(II), and Dy(II) are incorporated through covalent or non-covalent association, to complexing agents, including lipophilic derivatives, or to proteinaceous marcomolecules. Other magnetically active agents such as paramagnetic, superparamagnetic, ferrormagnetic agents, or the variations of them that enhance magnetic susceptibility may also be incorporated within the oil layer when alkylated or combined with other derivatives. The magnetically active agents are then dissolved in the oil or wax with the partition coefficient greater than 10. This composition is then added or dispersed into an aqueous phase containing one or more surfactant and stabilizing media. This composition is placed in a vial and is sealed with a head space of a preselected gas. The vial is then shaken for 45 seconds on an Espe CAPAMIX dental amalgamator at 4500 RPM.

This process results in liospheres containing magnetically active agents in the oil layer with a central gas bubble. Refer to FIG. 6C.

Microbubble Specifications: Size, Gas Type, and Biodistribution

The ideal microbubble contrast system should be made with a gas that has a solubility that is less than that of nitrogen so as to remain stable In circulation within the imaging time. Gaseous precursors that change state from liquid to gas due to shaking or that are temperature sensitive such as those composed of perfluorocarbon or those gas containing microbubbles in native state in room temperature are ideal choices. Microbubbles should be formed in a manner that biodistribution be narrowly distributed between 4-10 μm with a mean diameter of approximately 4-6 μm. The aforementioned prior art describe in detail the specific composition of lipid, volume of gas in the headspace of the vial, ideal surfactant, the duration and the vigor of shaking, and the filtration techniques that are necessary to make the desired formulations.

Microbubble and Nanoparticle Toxicity

When considering the toxicity associated with the proposed contrast agent system one needs to consider two different sources of toxicity microbubble toxicity and the toxicity of the magnetic agents that get chelated/embedded onto the surface of the microbubbles.

(A). Lipid-Shelled Microbubble Toxicity

The consensus among experts on high doses of microbubbles (in excess of 1 cc/kg of body mass) is quite varied. Alexander et al note that since LD50 of these contrast agents in mice are above 15 cc/kg and they expect 1 cc/kg would not cause any physiological complications in humans. Another group has found physiological complications start to emerge after an administration of 0.3 cc/kg with the primary complication being reduced systolic and diastolic pressure levels as reported by N C Nanda et al in “Echo-enhancing Agents: safety”. In: N Nanda et al eds. Advances in echo imaging using contrast enhancement, Dubai:Kluwer academic publishers; 1997. p 115-131. In other studies involving lipid coated microbubbles, Phase I clinical studies have shown that 0.15 cc/kg was safe and well tolerated by all subjects as reported by T A Fritz et al in “Phase I clinical trials of MRX-115; A new ultrasound contrast agent, Inves Radiol 1997; 32:735-740. With the contrast agents the inventors propose for pressure measurements it should be possible to produce microbubble contrast agents that can be sensitive even when the doses are below 0.30 cc/kg.

(B). Nanoparticle Toxicity

We have identified a number of different superparamagnetic agents that in theory can be chelated/embedded onto the lipid shells of the microbubbles. However, we choose to use Magnetite (Fe₃O₄) or the fully oxidized form of magnetite—maghemite (γ-Fe₂O₃) as they have already seen clinical use in MRI. In an earlier work, for sensitive detection of pressure, we showed that be in excess of 34 ppm in SI units at imaging the field strength of 1.5T with microbubble dose of 0.87 cc/kg is required. Our calculations to date show that of 50 ppm (SI) at a microbubble dose of 0.17 cc/kg can be obtained when superparamagnetic magnetite particles of radius 15 nm are dispersed in lipid shell at a shell volume fraction (defined as the ratio of total volume of the particles to volume of shell) of 1.02%. This is tantamount to uniformly dispersing approximately 2350 magnetite particles on each of the nearly 8.2 billion lipid shelled medical grade bubbles of 2 μm radius. This coating is equivalent to a total iron dose of 1.8 mg that is well below the dose (in excess of 280 mg) at which physiological complications emerge as reported by M Taylor et al in “Safety and prelimiuary findings with the intravascular contrast agent NC100150 injection for M coronary angiography, JMRI 1999; 9:220-227.

Dose dependence on Measurement Accuracy in R2 for MR Manometry

As pointed out earlier, the measured

$${R}_{2}^{Blood}$$

in the presence of microbubble will be a combination of R2 due to dipole-dipole coupling and diffusion through local field inhomogeneities that is dependent on the oxygen state of the blood and the presence of micobubbles. If we can detect the changes in

$${R}_{2}^{Bubb}$$

perfectly, to detect a pressure change of ΔP with 95% confidence subject to an error of a in R2 of blood without bubbles (R2^{I}), it can be shown that

$${R}_{2}^{Bubb} \geq \frac{2 \cdot σ \cdot {R}_{2}^{I}}{k \cdot Δ P} , \qquad ( 12 )$$

where k is the relative change in

$${R}_{2}^{Bubb}$$

due to change in pressure. From our calculations, we observed k≈0.066 mmHg⁻¹. Hence, to detect a pressure change of 50 mmHg above atmospheric pressure the minimum necessary

$${R}_{2}^{Bubb}$$

will be related to the measurement accuracy of R2^{I}. Table

TABLE 2
$\begin{matrix}Dependence of measurement accuracy of {R}_{2}^{I} on \\ {R}_{2}^{Bubb} for sensitive detection of moderate pulmonary hypertension\end{matrix}$

| | |
|---|---|
| $\begin{matrix}Percent accuracy in the \\ measurement of {R}_{2}^{I} ( σ )\end{matrix}$ | ${minimum}^{a} {R}_{2}^{Bubb}$ |

| | |
|---|---|
| 5 | 14 |
| 4 | 11 |
| 3 | 8.6 |
| 2 | 5.7 |
| 1 | 2.9 |

(i). as predicted by Eq.(12)

2 lists the minimum

$${R}_{2}^{Bubb}$$

values needed to detect 50 mmHg pressure change to the atmospheric pressure when 0.01≦σ≦0.05. As σ decreases,

$${R}_{2}^{Bubb}$$

also decreases indicating that as the measurement accuracy of R2ⁱ increases, the microbubble dose necessary to make the measurement can be decreased further.

Microbubble Based In Vivo MR Manometry

Microbubble based MR Manometry relies on the intravenous delivery of microbubble contrast media, a calibration curve for a given contrast agent at a given dose between pressure and R2^{Blood}, a flow independent and motion compensated MRI protocol to measure R2^{Blood} in a region where the gauge pressure is approximately zero and in the region of interest where the pressure is to be measured. Calibration curve between the the ambient pressure and R₂^{Blood} can be established for all physiological doses of interest with the aid of a catheter for a given contrast agent. Following this, a physiologically tolerable dose of sensitivity enhanced microbubble contrast media is delivered intravenously either as a bolus or as a continuous infusion., The passage towards steady state microbubble concentration can be monitored by measuring the MR signal changes in a large vein such as the brachiocephalic vein where the gauge pressures are nearly zero. Following this a similar MR pulse sequence as outlined in the U.S. Pat. No. 6,094,591 which is incorporated herein by reference in its entirety, be used to measure Intracardiac or vascular R₂^{Blood} in the presence of the microbubble contrast media.

The prior art pulse sequence begins with a 90ₓ excitation pulse followed by a train of 180_{y} refocusing pulses, which are equally separated by a refocusing interval termed τ₁₈₀. Spatial localization is performed using a final slice-selective pulse followed by an imaging gradient. To measure R2^{Blood}, a series of T2-weighted images is acquired with this pulse sequence in which the duration of the refocusing train is set to different values by changing the number of refocusing pules used. With these images, R₂^{Blood} can be estimated by extracting the signal amplitude within the blood vessel and fitting the data points as a monoexponential decay using a weighted least squares fit.

To minimize flow sensitivity when using this pulse sequence, the excitation pulse and refocusing train axe non-selective. Thus, there are no gradients applied and no moments to be nulled. In addition, the regular refocusing achieved by the train of 180_{y} pulses lessens the amount of dephasing due to flow through susceptibility gradients. Assuming ideal RF homogeneity, phase accrued by spins moving at a constant velocity through local B₀ inhomogeneity can be modeled as a linear gradient. The validity of such a model improves as τ₁₈₀ decreases because spine travel a shorter distance between pulses.

In this implementation of a T2 weighted magnetization preparation the T2-weighted magnetization produced by the train of 180_{y} refocusing pulses is returned to the longitudinal axis at the echo of the final refocusing pulse. Manipulation of T2 contrast from the transverse plane back to the longitudinal axis is achieved using a 90₋ₓ tip-up pulse. At this time, a spoiler gradient is also applied along the slice-select axis to dephase any residual transverse magnetization.

The principal advantage of temporary longitudinal storage of T2 contrast using the present invention is the flexibility it allows in the choice of imaging pulse sequences. For example, in one embodiment the T2 preparation segment is followed by an imaging pulse sequence in which a series of tip-up angle RP excitations follow the tip-up RF pulse at the completion of the T2 preparation segment. Different slices or different part of k-space may be acquired after each small tip age RF excitation pulse. In the preferred embodiment described below, a single slice imaging pulse sequence is used in which a spectrally and spatially selective RF excitation pulse and spiral interleaf readout is employed. Because the spectral-spatial RF pulse selectively excites water while isolating the slice of interest, this sequence rejects lipids. The spiral acquisition is well-suited for vascular imaging due to its excellent flow properties.

In addition to tipping the T2-weighted magnetization back into the longitudinal axis, the T2 preparation segment addresses a number of important issues. The effects of RF and static field inhomogeneities during the refocusing train are handled using trains of relatively simple composite refocusing pulses with good RF cycling patterns. It is preferred that a MLEV pattern of 90ₓ-180_{y}-90ₓ composite refocusing pulses is used and all pulses are rectangular and non-selective with γB₁/2 π<1 kHz. When using composite refocusing pulses, methods are used to compensate for T1 signal decay effects during each refocusing pulse. Solutions include decreasing the pulse duration, increasing the refocusing interval, and using post-processing methods. It is that one uses a simple shift of echo times to account for T1 signal decay effects without constraining the pulse duration or the refocusing interval.

The effects of RF field offsets on the 90ₓ/90₋ₓ excitation/tip-up pulse pair is addressed by using phase-cycling methods which subtract out the T1 bias or by using composite 900 excitation and tip-up pulses which ensure an efficient manipulation of magnetization between the transverse plane and the longitudinal axis. It is preferred one uses a 360ₓ-270ₓ-90_{y} pulse for excitation and a 45-x-90_{-y}-90-x-45_{y} pulse for tip-up. This pulse combination provides dual RF and static field insensitivity without increasing the imaging time.

Following the preparation interval, T2 contrast is stored temporarily along the longitudinal axis. During this time, the T2-weighting will degrade gradually by T1 relaxation effects. Methods which remove the additive T1 recovery term will preserve the prepared T2 contrast. The preferred embodiment cycles the longitudinally-stored T2 contrast between the ±z axes by applying a robust inversion pulse immediately following the tiwup pulse on subsequent excitation. The additive term is removed upon subtraction of the acquired data. When using a series of small-tip angle excitations, the sensitivity to subtraction errors can be reduced by applying an inversion pulse following each small-tip angle excitation.

Due to the strong dependence of the contrast agent effect on τ₁₈₀, careful selection of this parameter is an important aspect of the oximetry protocol. Hence an optimal τ₁₈₀ that sufficiently balances the contrast that can be developed within this time yet one that reduces the flow artifacts for a given microbubble dose and Δχ is necessary. It is preferred that τ₁₈₀ of 6 ms or smaller is used to optimize the pressure contrast based on R2^{Blood}.

A signal-to-noise ratio per pixel greater than 10 at the time of the longest T2 preparation interval is essential to avoid noise bias in the R2^{Blood} measurement. In the large vessels, which are closer to the body surface, this SNR is achieved easily using a conventional 5 inch surface coil. Due to the rapid drop-off of sensitivity with depth when using such a coil, the SNR/pixel may be prohibitively low for measurements in small and centrally located vessels, such as those in and around the heart. To overcome this problem it is desirable to use an array of local coils to receive the MR signal. Good visualization of a vessel for Manometry requires adequate spatial resolution and an imaging slice which is perpendicular to the vessel wall. This is straightforward for measurements in large vessels with little motion. Measurements within smaller vessels which move considerably, such as those in and around the heart, pose a greater challenge for reliable visualization. Spatial resolution can be increased by sampling higher spatial frequencies during the data acquisition. The preferred method is to place at least 6 pixels across the vessel diameter. In addition, the SNR limitation also require that microbubble dose do not exceed the limits of detection so as to create pockets of “black-blood” in the images. Hence, it is imperative pressure sensitive MR contrast is developed in a manner that limits dose both due to toxicity limitations and due to measurement limitations.

R₂^{Blood} measurements in and around the heart axe inherently sensitive to respiratory motion due to the relatively long data acquisition times. If not compensated for, blurring and motion artifacts will degrade the quality of each T2-weighted image. A number of respiratory compensation methods exist which can improve image quality. Schemes which rely on breath-holding, rapid imaging, or motion monitoring and reacquisition methods attempt to reduce the number of respiratory phases in the acquired data Other methods rely on the periodicity of the respiratory cycle to implement post-processing corrections.

It is preferred that one uses a respiratory bellows and the signal processing unit of the MR imager to monitor and record the respiratory phase at the time of each data acquisition. Following the collection of a full data set, a histogram of the respiratory phases is constructed. Overscanning and the well-known Diminishing Variance Algorithm are then applied to “freeze” the respiratory motion. In addition, if not compensated, cardiac motion can introduce considerable artifacts and blurring into a T2-weighted image. Methods to “freeze” heart motion rely on prospective gating using a plethysmograph placed on a finger for an ECG trigger. Due to the considerable delay between the R wave and the triggering of the plethysmograph, the preferred embodiment uses the R wave of the ECG signal for triggering the pulse sequence.

Because the acquisition of T2-weighted images in and around the heart requires multiple data acquisitions, a steady-state longitudinal magnetization is desirable at the time of each excitation. For vascular T2 measurements, a steady-state magnetization is difficult to achieve due to variability in the heart rate. The simplest method to reduce the effects of heart rate variability on the R2^{Blood} measurement is to allow more than one heart beat for T1 recovery. Other methods control the duration of T1 recovery by nulling the longitudinal magnetization at a set time before each excitation pulse. To overcome this problem it is desirable to acquire data following every other heart beat.

By collecting the R2^{Blood} at pressure independent region such as the brachiocephalic or jugular vein and the region of interest where the pressure is to be measured, and computing the differences between the respective R2^{Blood}s and using the aforementioned calibration curve, pressure in a region of interest is mapped.

As used herein, the terms “comprises”, “comprising”, “including” and “includes” are to be construed as being inclusive and open ended, and not exclusive. Specifically, when used in this specification including claims, the terms “comprises”, “comprising”, “including” and “includes” and variations thereof mean the specified features, steps or components are included. These terms are not to be interpreted to exclude the presence of other features, steps or components.

The foregoing description of the preferred embodiments of the invention has been presented to illustrate the principles of the invention and not to limit the invention to the particular embodiment illustrated. It is intended that the scope of the invention be defined by all of the embodiments encompassed within the following claims and their equivalents.

### OTHER PUBLICATIONS

Rich S. Braunwald E, Grossman W. Pulmonary hypertension. In: Braunwald E, editor. Heart Disease, 5th edition. Philadelphia: W. B. Saunders Compnay; 1998. p 780-806.

Berger M, Haimowitz A, Tosh AV. Quantitative assessment of pulmonary hypertension in patients with tricuspid regurgitation using continuous wave Dopplerultrasound. Am J Cardiol 1985; 6:359-365.

Bouchard A, Higgins C B, Byrd, B F. Magnetic resonance imaging in pulmonary hypertension. Am J Cardiol 1985;56: 938-942

Urchuk S N, Plewes D B. MR measurement of time-dependent blood pressure variations. J Magn Reson Imag 1995;5:621-627

Raeside D, Peacock, A. Making measurements in the pulmonary circulation: when and how?. Thorax 1997; 52:9-11.

Kircher B J, Himelman R B, Schiller N B. Noninvasive estimation of right atrial pressure from the inspiratory collapse of the Inferior vena cava. Am J Cardiol 1990;66:493-496

Fairbank W M, Scully M. A new noninvasive technique for cardiac pressure measurement: resonant scattering of ultrasound from bubbles. IEEE Trans Biomed Eng 1907; BME 24:107-110

Tickner E G. Precision Micro-bubbles for right side intracardiac pressure and flow measurements. In: Meltzer R S, Roelandt JTCR, eds. Contrast Echocardiography. Vol. 15. London: Martinus Nijho.,

Bouakaz A, Frinking P J A, Bom N. Noninvasive measurement of the hydrostatic pressure in fluid-filled cavity based on the disappearance time of micrometer-size free gas bubbles. Ultrasound Med Bio 1999. 25:1407-1415.

Alexander A L, McCreery T T, Barrette T R, Gmitro A F, Unger E. Microbubbles as novel pressure-sensitive MR contrast agents. Magn Reson Med 1996. 35:801-806

Wright G A, Hu M, Macovski A. Estimating oxygen saturation of blood in vivo with MR imaging at 1.5T. J Magn Reson Imag 1991;1: 275-283

Nanda N C, Cartensen E L. Echo-enhancing Agents: safety. In: Nanda N, Schlief R, Goldberg B B, eds. Actvances in echo imaging using contrast enhancement, 2nd edition. Dubai:Kluwer academic publishers; 1997. p 115-131

Dharmakumar R, Plewes D, Wright G A. On the parameters affecting the sensitivity of MR measures of pressure with microbubbles. Magn Reson Med 2002. 47: 264-273.

Glasel J A, Lee K H. On the interpretation of water nuclear magnetic resonance relaxation times in heterogeneous systems. J Am Chem Soc 96:970 (1974).

Dunlop D J, Ozdamir O. Rock magnetism: fundamental and frontiers. Cambridge University Press, 1997. p.51 and 131.

Fritz T A, Unger E C, Sutherland G, Sahn D. Phase I clinical trials of MRX-115: A new ultrasound contrast agent. Inves Radiol 1997; 32:735-740

Taylor M, Panting J R, Keegan J, Gatehous P D, Jhooti P, Yang O Z, McGill S, Burman ED, Francis J M, Firmin D N, Pennell D J. Safety and preliminary findings with the intravascular contrast agent NC100150 injection for MR coronary angiography. J Magn Reson Imag 1999; 9:220-227.

Therefor what is claimed is:

1. A microbubble for use in sensitivity enhanced magnetic resonance manometry, comprising a lipid shell having a high magnetic susceptibility.

2. A microbubble for sensitivity enhanced magnetic resonance manometry, comprising a lipid shell including magnetic nanoparticles having high dipole moments embedded therein.

3. The microbubble according to claim 2 wherein said lipid shell includes a substantially continuous coating of said magnetic nanoparticles.

4. The microbubble according to claim 2 wherein said magnetic nanoparticles are uniformly distributed over the surface of said lipid shell.

5. The microbubble according to claim 2 wherein said magnetic nanoparticles are non-uniformly distributed over the surface of said lipid shell.

6. The microbubble according to claim 2 wherein a preselected volume fraction of the magnetic nanoparticles are present on the microbubble shell for reducing the microbubble dose well below 1 cc/kg.

7. The microbubble according to claim 1 that are stabilized by encapsulating gases of low permeability across the lipid membrane.

8. A microbubble for sensitivity enhanced magnetic resonance manometry, comprising a lipid shell including a magnetically active agent attached to, or incorporated into, the surface of the bubble to give said microbubble a preselected magnetic susceptibility.

9. A use of coated microbubbles to decrease microbubble dose necessary to detect a desired pressure change in the circulation by improving the measurement accuracy of the MR signal decay rate constant related to blood oxygen effect and dipole-dipole coupling of water protons.

10. A magnetic resonance imaging method for measuring intravascular or intracardiac pressure in a patient, the method comprising the steps of;
a) intravenously administering microbubbles to a patient, said microbubbles comprising a lipid shell having a high magnetic susceptibility;
b) performing cardiac-gated, flow and/or motion compensated magnetic resonance imaging to establish microbubble concentration dependent and pressure independent magnetic resonance (MR) signal decay in a major blood vessel or in a sample of blood drawn from said patient; and
c) measuring the magnetic resonance signal in a region of interest in the patient's body, comparing a difference between pressure independent magnetic resonance signal and pressure dependent magnetic resonance signal to a calibration curve between magnetic resonance signal decay and pressure to determine the pressure in the region of interest.

11. The method according to claim 10 wherein said major blood vessel is the brachiocephalic vein or a vein where the pressure is nearly zero relative to atmospheric pressure.

12. The method according to claim 10 wherein said region of interest in the patient's body is the patient's cardiac chamber or a selected part of the patient's vascular system.

13. The method according to claim 10 wherein the step of performing cardiac-gated, flow and/or motion compensated magnetic resonance imaging includes applying a pulse sequence beginning with a 90ₓ excitation pulse followed by a train of 180_{y} refocusing pulses, which are equally separated by a refocusing interval termed τ₁₈₀, performing spatial localization using a final slice-selective pulse followed by an imaging gradient, acquiring a series of T2-weighted images with the pulse sequence in which the duration of the refocusing train is set to different values by changing the number of refocusing pulses used, and estimating R2^{Blood} by extracting the signal amplitude within the blood vessel and fitting the data points using an effective function.

14. The method according to claim 13 wherein the effective function is a monoexponential decay function using a weighted least squares fit.

15. The method according to claim 13 wherein the excitation pulse and refocusing train are non-selective to minimize flow sensitivity when using this pulse sequence whereby substantially no gradients applied so that no moments to be nulled.

16. The method according to claim 13 wherein in the implementation of a T2-weighted magnetization preparation the T2-weighted magnetization produced by the train of 180_{y} refocusing pulses is returned to a longitudinal axis at the echo of the final refocusing pulse, and wherein manipulation of T2 contrast from the transverse plane back to the longitudinal axis is achieved using a 90-x tip-up pulse, including at this time applying a spoiler gradient along the slice-select axis to dephase any residual transverse magnetization.

17. The method according to claim 16 wherein the T2 preparation segment is followed by an imaging pulse sequence in which a series of tip-up angle RF excitations follow the tip-up RF pulse at the completion of the T2 preparation segment.

18. The method according to claim 13 wherein different slices or different parts of k-space may be acquired after each small tip angle RF excitation pulse.

19. The method according to claim 18 wherein a single slice imaging pulse sequence is used in which a spectrally and spatially selective RF excitation pulse and spiral interleaf readout is employed whereby because the spectral-spatial RF pulse selectively excites water while isolating the slice of interest, this sequence rejects lipids.

20. The method according to claim 16 wherein the refocusing pulse trains comprise a pattern of 90x-180y-90x composite refocusing pulses wherein all pulses are rectangular and non-selective with γB₁/2 π<1 kHz.

21. The method according to claim 20 wherein when composite refocusing pulses are used, including compensating for T1 signal decay effects during each refocusing pulse.

22. The method according to claim 21 wherein compensating for T1 signal decay effects during each refocusing pulse includes one or more of decreasing the pulse duration, increasing the refocusing interval, or using post-processing methods.

23. The method according to claim 21 wherein effects of RF field offsets on the 90ₓ/90₋ₓ excitation/tip-up pulse pair is compensated for by using phase-cycling methods which subtract out a T1 bias or by using composite 90° excitation and tip-up pulses which ensure an efficient manipulation of magnetization between the transverse plane and the longitudinal axis.

24. The method according to claim 23 wherein said excitation pulse is 360ₓ-270ₓ-90_{y} are used as excitation pulses and 45-x-90_{-y}-90-x-45_{y} pulses are used for the for tip-up pulses for providing dual RF and static field insensitivity without substantially increasing imaging time.

25. The method according to claim 23 wherein following the preparation interval, T2 contrast is stored temporarily along the longitudinal axis, including removing the additive T1 recovery term to preserve the prepared T2 contrast by cycling the longitudinally stored T2 contrast between the ±z axes by applying an inversion pulse immediately following the tip-up pulse on subsequent excitation, and including removing the additive term upon subtraction of the acquired data.

26. The method according to claim 16 wherein τ₁₈₀ is 6 ms or less.

27. The method according to claim 16 wherein a signal-to-noise ratio per pixel greater than 10 at the time of the longest T2 preparation interval is used to avoid noise bias in the R2^{Blood} measurement.

28. The method according to claim 16 wherein when measuring R2^{Blood} in larger blood vessels closer to the body surface, step c) of measuring the magnetic resonance signal includes using a 5 inch surface coil for receiving the MR signal.

29. The method according to claim 16 wherein when measuring R2^{Blood} in smaller blood vessels centrally located in the body surface, step c) of measuring the magnetic resonance signal includes using an array of coils for receiving the MR signal.

30. The method according to claim 10 wherein a respiratory bellows is used and a signal processing unit of a magnetic resonance (MR) imager is used to monitor and record a respiratory phase at a time of each data acquisition.

31. The method according to claim 30 wherein following collection of a full data set, a histogram of the respiratory phases is constructed, and wherein overscanning and using a Diminishing Variance Algorithm are then applied to “freeze” the respiratory motion.

32. The method according to claim 30 including compensating for cardiac motion by gating using a plethysmograph placed on a finger of the patient for an ECG trigger, and wherein an R wave of the ECG signal is used for triggering the pulse sequence.

33. The method according to claim 30 including using a steady-state longitudinal magnetization at the time of each excitation and acquiring data following every other heart beat.

34. The method according to claim 10 wherein including collecting the R2^{Blood} at a pressure independent region such as the brachiocephalic or jugular vein and the region of interest where the pressure is to be measured, and computing the differences between the respective R2^{Bloods} and using the aforementioned calibration curve, pressure in a region of interest is mapped.
